(19) United States
(12) Patent Application Publication
(10) Pub. No.: US 2006/0004340 A1
(43) Pub. Date: Jan. 5, 2006

(54) Anti-irritant disposable diaper

(76) Inventor: Chaim Ben-Natan, Kfar Habad (IL)

Correspondence Address: Edward Langer;c/o Shiboleth, Yisraeli, Roberts, Zisman & Co., 60th Floor, 350 Fifth Avenue, New York, NY, 10118, US

(21) Appl. No.: 11/108,074

(22) Filed: Apr. 18, 2005

Related U.S. Application Data
(60) Provisional application No. 60/584,305, filed on Jun. 30, 2004.

(51) Int. Cl. A61F 13/15

(52) U.S. Cl. 604/385.19

(57) ABSTRACT

An improved, anti-irritant, disposable diaper. The diaper is comprised of a front portion, a crotch area, a rear portion, closing tabs for securing the diaper upon a wearer, waste containment means for collecting and isolating body wastes; attachment means for detachably attaching said containment means to the crotch area of the interior said diaper. The improvement comprises a reinforced, semi-rigid ribbing disposed contiguous to at least one surface of the containment means, for restoring the containment means to its original shape after being compressively deformed, and closure means provided on the containment means, to ensure closure of the containment means when the wearer is seated.

### FIELD OF THE INVENTION

The present invention relates to disposable diapers, in particular to diapers constructed to collect and isolate body wastes so as to protect wearers from skin irritation.

### BACKGROUND OF THE INVENTION

In my previous U.S. Pat. No. 6,464,673 issued Oct. 15, 2002, there was disclosed an anti-irritant disposable diaper designed to contain body waste and maintain it and wet absorbent material apart from the skin of an infant, thus removing the two major sources of irritation caused by prior art disposable diapers.

The present invention improves on the prior art by providing a more economical and efficient container for holding solid and urinary body wastes. The container of the present invention is designed so as to return to its original shape when deformed by body weight pressure being applied to it, especially when there is change of a user's position from standing to sitting on a rigid surface. Additionally, the present invention provides closing means which ensure the container is held closed during sitting, so that the contents of the container do not leak out during sitting on a rigid surface. The present invention introduces features not heretofore anticipated and therefore novel and also not obvious.

Use of disposable diapers for both infants and incontinent adults, especially geriatric patients, has provided a convenient and sanitary solution for those responsible for daily care and personal hygiene of wearers of diapers. However, the plastic coating used on the outside of the absorbent layers, while helping to prevent leaks, also causes body wastes to be retained in close contact with the skin of the wearer, and creates a breeding ground for rash-causing micro-organisms.

Many advances have been made in diaper technology such as the use of gels to lock in liquid waste and prevent it from irritating a wearer's skin. These advances have improved the use of disposable diapers. However, many wearers continue to suffer from skin irritations and rashes. A more apparent problem is those rashes caused by contact with solid body waste.

Additionally, disposable diapers can be expensive to use and are perceived as wasteful. Dirty diapers must be changed as soon as possible so as to prevent rashes, but often the diaper is not wet and its absorbent capacities have not been realized. Because disposable diapers are not biodegradable, it is necessary to find ways of conserving the use of diapers.

Therefore, it would be desirable to provide a method of containing body wastes, to prevent their coming into contact with the skin of a wearer, so as to avoid rashes. This would also ease cleaning a wearer during changing of the diaper. It would also be desirable to provide for an economic use of disposable diapers so that diapers that have not been used to their full potential may be re-used.

### SUMMARY OF THE INVENTION

Accordingly, it is a principal object of the present invention to overcome the problems of prior art diapers, and provide an improved, anti-irritant, disposable diaper.

In accordance with a preferred embodiment of the present invention, there is provided an improved disposable diaper comprised of a front portion, a crotch area, a rear portion, closing tabs for securing the diaper upon a wearer, waste containment means for collecting and isolating body wastes; attachment means for detachably attaching said containment means to the crotch area of the interior said diaper; wherein the improvement comprises:
a reinforced, semi-rigid ribbing disposed contiguous to at least one surface of said containment means, for restoring said containment means to its original shape after being compressively deformed; and closure means provided on said containment means, to ensure closure of said containment means when the wearer is seated.

In the present invention the reinforced semi-rigid ribbing maintains the distinctive shape of the containment means, such as in the form of a four-sided, open, flexible nylon sack or bag, while not causing discomfort or injury to the wearer of the diaper. The ribbing provides for a controlled folding of the sack when a wearer is seated, in such a way as to contain any waste matter therein. The semi-rigid ribbing on the containment means maintains it in a fixed, open position while the wearer is standing.

During sitting, it has been found that leakage of solid waste out of the opening of the waste sack must be prevented, therefore the present invention provides closure means to ensure closure of the waste containment sack during sitting on a rigid surface. In the most preferred embodiment, the semi-rigid ribbing acts as the closure means, since the ribbing has at least one region of lesser rigidity, such as pre-creases, upon which the ribbing may fold and close the neck of the containment sack. According to this embodiment, during sitting, pressure is exerted from the ground or chair, through the semi-rigid ribbing, onto the upper lip of the containment means, causing the sides of the containment means to be forced outward or inward at right angles to the vertical axis of the containment means. This folding is controllable by pre-creasing the semi-rigid ribbing either on an interior or exterior side, respectively, in relation to the orientation of the ribbing to the containment means.

The containment means, in another embodiment of the invention, folds on only one side or in one direction depending on the orientation and disposition of supporting ribs on one or more of the surfaces of the containment means. In either case, the folds generally narrow an area of the containment means to prevent wastes from accidentally escaping and coming into contact with the skin of a wearer. Even with a diaper formed from absorbent material, portions of the diaper that lie closest to the skin of a wearer might be subject to excessive amounts of body waste from seepage out of the container. The present invention accounts for this by providing closing that shuts the opening of the container against accidental seepage when the container is compressed and allows for the container to naturally resume its full volume state when the sitting pressure is removed.

According to a preferred embodiment, the containment means, such as a nylon sack, can be attached directly to the diaper itself, in the crotch area so that the sack cannot become twisted or displaced and thereby accidentally release its contents.

In a case where a diaper is dirty and not wet, it would only be necessary to open the diaper, remove the sack and replace it. This would conserve for reuse a diaper with absorbent padding which has not been used for liquid waste; overall, a considerable saving in cost of new diapers.

In one embodiment, the waste containment means can be provided with a flexible flap extending from the front portion of the crotch area of the diaper and being positioned under the wearer so as to cover the waste when the wearer is sitting down and to withdraw when the wearer resumes a standing or upright position.

In an alternative embodiment of the invention, the semi-rigid ribbing is slidable upon at least one pair of slide tracks which retains a partially-attached, flexible, extended tongue piece. When a wearer sits down, pressure from the body forces the tongue piece, which is attached only at the lower-most surface of a containment means, such as a sack, to slide upward along the tracks, closing the opening. In effect, the folded sides of the sack move with the tracks down over the lower section of the tongue piece so that the upper section which is not attached, is guided to form a closure for the opening of the sack.

Upon resuming a standing position, the pressure on the sack is released and the sack resumes its initially opened form assisted by the expansion of the compression folds and the latent energy of the semi-rigid plastic tracks which produce a small, but significant counter-force to the compression. This expansion movement of the folds caused by decompression, as well as the small counter-force of the tracks, causes the slide tracks, which are attached to the folds, to move in such a way as to relatively encompass more of the tongue piece, in effect, exposing the opening which once again allows wastes to collect in the sack.

In another embodiment, instead of a single tongue piece, the containment means is provided with two pairs of tracks attached to the outer bottom portion of the diaper and two tongue pieces which slide within the tracks and which are oppositely disposed from one another, in relation to the opening, and designed to meet and close the opening to the containment means as described above for the single tongue piece. The tongue pieces extend from the bottom of the containment means along the tracks so that when a diaper user sits on the containment means, the tongues are forced to come together and overlap the opening in the diaper, effectively preventing body wastes from spilling out of the containment means and causing skin irritation and discomfort to the diaper user. The two tongue pieces slightly overlap to provide a secure closure and easily separate when the pressure on the containment means is relieved.

The advantage of two tongue pieces is that the length of the tongue pieces can be shortened and the closing and opening action occurs over a shorter distance, thus more quickly protecting the wearer from accidental spill-out of wastes from the containment means when sitting.

Other features and advantages of the invention will become apparent from the following drawings and descriptions.

### BRIEF DESCRIPTION OF THE DRAWINGS

For a better understanding of the invention with regard to the embodiments thereof, reference is made to the accompanying drawings, in which like numerals designate corresponding elements or sections throughout and in which:

FIG. 1 shows a pictorial view of a diaper fitted with a waste containment means of the present invention;

FIG. 2A shows an orthographic view illustrating an over-all view of an embodiment of the containment means of the present invention;

FIG. 2B shows an isometric side view of the containment means of FIG. 2A in a compressed mode when the wearer is in a sitting position; and

FIGS. 3A-C are various views of another embodiment of the containment means of the present invention.

### DETAILED DESCRIPTION OF THE INVENTION

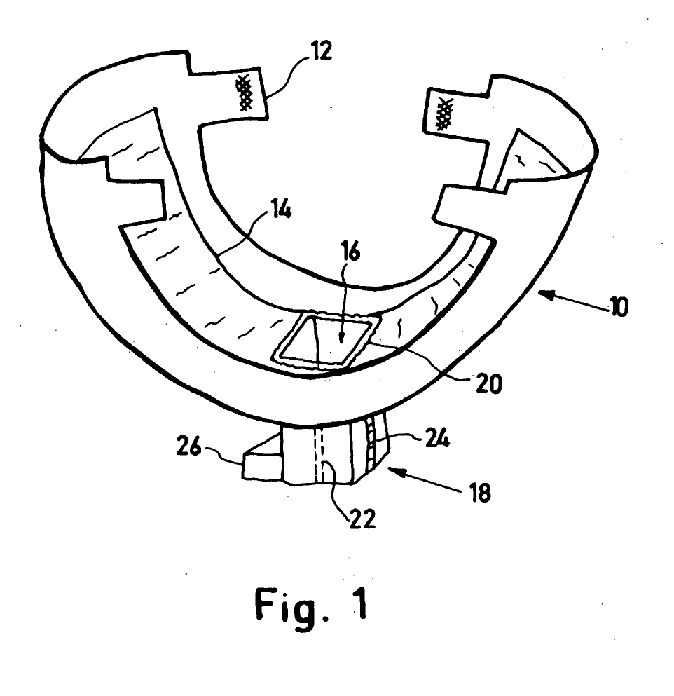

Referring now to FIG. 1 there is shown diaper 10 constructed in accordance with the principles of the present invention. Diaper 10 comprises four tabs 12 extending from the extreme ends of the front and back portions of the diaper which enable attaching and detaching the diaper 10 from a wearer for changing when necessary. A standard absorbent layer 14 is provided for absorbing urinal discharges. An opening 16 is provided in a central crotch position for allowing solid body wastes, and excess, unabsorbed urinal discharges, to exit through opening 16 in diaper 10 and be deposited in a containment means 18, such as, for example, a nylon bag or sack. Containment means 18 may be permanently affixed to diaper 10, or detachable. As shown in a preferred embodiment of the invention, opening 16 is substantially rectangular, but other shapes, such as oval or round, may also be provided.

Containment means 18, such as a nylon sack or bag, is disposed beneath opening 16 that is situated in the approximate crotch area of diaper 10. Containment means 18 (unless otherwise indicated, hereinafter referred to as sack 18) is attached to diaper 10 by pressing the edge of sack 18 which lies along the perimeter of opening 16 against a matching adhesive area 20 provided on diaper 10. When the wearer is seated, sack 18 becomes folded in such a way as to contain body wastes discharged into sack 18.

In one embodiment of the invention, sack 18 is placed over the hand and inserted through opening 16 in the crotch area of diaper 10, such that the perimeter of the open end of sack 18 are brought into proximity to the upper edge of opening 16. Then the upper portion of the open end of sack 18 is pressed firmly against the adhesive area 20. Sack 18 is positioned directly under opening 16 and attached to diaper 10 via attachment means 20, such as an adhesive edge. Sack 18 is a container generally four-sided in shape in a preferred embodiment of the invention, but other shapes are also possible.

The attachment area 20 is optionally disposed either under or over the opening 16, but in a preferred embodiment of the invention, sack 18 is inserted from inside the diaper 10 to provide additional strength to the attachment. The attachment may, alternatively, be any type of detachable attachment as is known to those skilled in the art, such as Velcro, sticky adhesive, and the like.

In a preferred embodiment of the invention, sack 18 is supported in an upright and open condition, by a semi-rigid ribbing 22 which closely wraps around opposing upright sides and bottom of sack 18 in a continuous band. Alternatively, sack 18 is provided with at least one additional support ribbing 24 which crosses the ribbing 22 on the bottom surface of sack 18 at right angles and wraps around the remaining two sides of sack 18 in a continuous band. The width of semi-rigid ribbing 22, 24 is calculated at a minimum so as to be suited to the physical size and weight of the wearer. Obviously an adult diaper would require a ribbing that is stronger and wider than that used for an infant diaper.

In another embodiment of the invention (not shown), the semi-rigid ribbing comprises the entire width of the bottom surface and two opposite sides of sack 18 extending upward in a continuous band to the crotch area thus forming a maximal width ribbing.

Preferably, the semi-rigid ribbing 22, 24 is pre-creased so as to compress outward, or, alternatively, inward, when a wearer assumes a sitting position and provided with such resiliency as to return to an uncompressed, fully extended shape when a wearer resumes a standing or upright position.

Optionally, an additional collecting chamber 26 is provided with sack 18 which functions as an overflow chamber for wastes for wearers in need of a larger capacity containment means. The sack 18 is designed for a general capacity of solid and urinal waste discharges in accordance with the physical dimensions of a wearer, whether an infant, a child, or an adult, so as not to cause a wearer discomfort. The collecting chamber 26 is manufactured as an extension of at least one of the front, back and both sides of sack 18 in such as way as to be positioned between the legs of the wearer.

In a sitting position, the wearer is not bothered by the slight extension of sack 18, yet body wastes are hygienically collected and kept apart from the skin of the wearer. Such extensions incidentally provide for longer use of sack 18 between changes of diaper 10 without creating a health hazard.

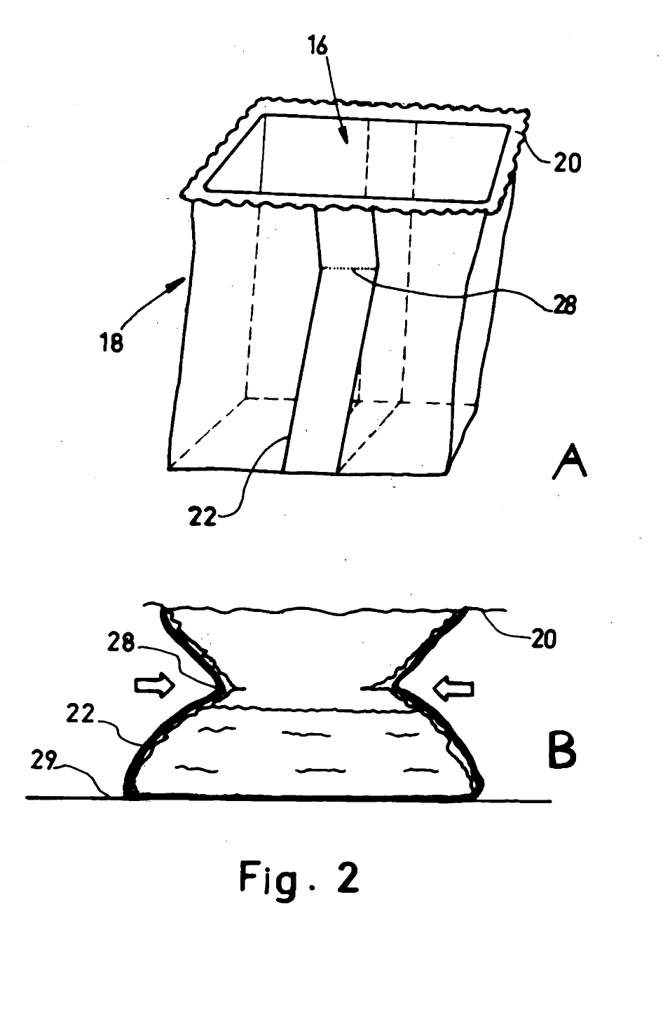

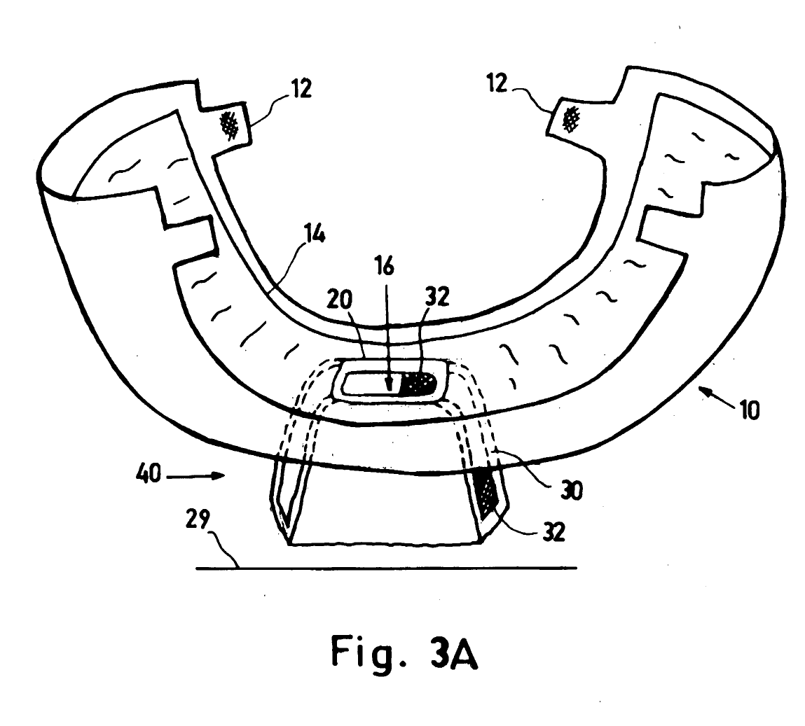

Referring now to FIGS. 2A and 2B, there is illustrated a detailed view of an embodiment of a body waste collection means of the present invention. The sack 18, such as a soft and flexible nylon sack, is lined on the bottom and at least on two opposite sides with a semi-rigid ribbing 22 which is pre-creased along crease line 28 so as to allow for folding of sack 18 when a wearer is seated. The folds form inward or outward depending on the placement of pre-creases such as crease line 28 in sack 18 when it is compressed between the body of the wearer and a firm seating surface, such as 29 in FIG. 2B.

The folds and flexible material of sack 18, made of soft nylon in a preferred embodiment of the invention, are able to resist tearing or bursting from the force of compression. Opening 16 is held open due to attachment around the perimeter of opening 16, but sack 18 becomes folded inwardly at the pre-creases formed in ribbing 22 so as to form a tight, narrow neck as shown by the horizontal arrows in FIG. 2B so as to prevent the pressure of the wearer on sack 18 to accidentally discharge the contents.

When sack 18 is soiled or overly full, of either liquid or solid body wastes, the tabs 12 (shown in FIG. 1) of diaper 10 are opened and sack 18 is removed. It is not necessary to replace the whole diaper 10 if the absorbent layer 14 is unsoiled, rather a new sack 18 replaces the old and diaper 10 is ready for further use. Sack 18 is provided with an absorbent layer 14 also, so that most if not all urinary discharges are quickly isolated and absorbed. The improved diaper of the present invention is economical, as besides isolating solid body wastes, it also collects all excess urinal discharges within sack 18 so that, if the larger area of absorbent material 14 lining diaper 10 (see FIG. 1) remains dry, it may be reused.

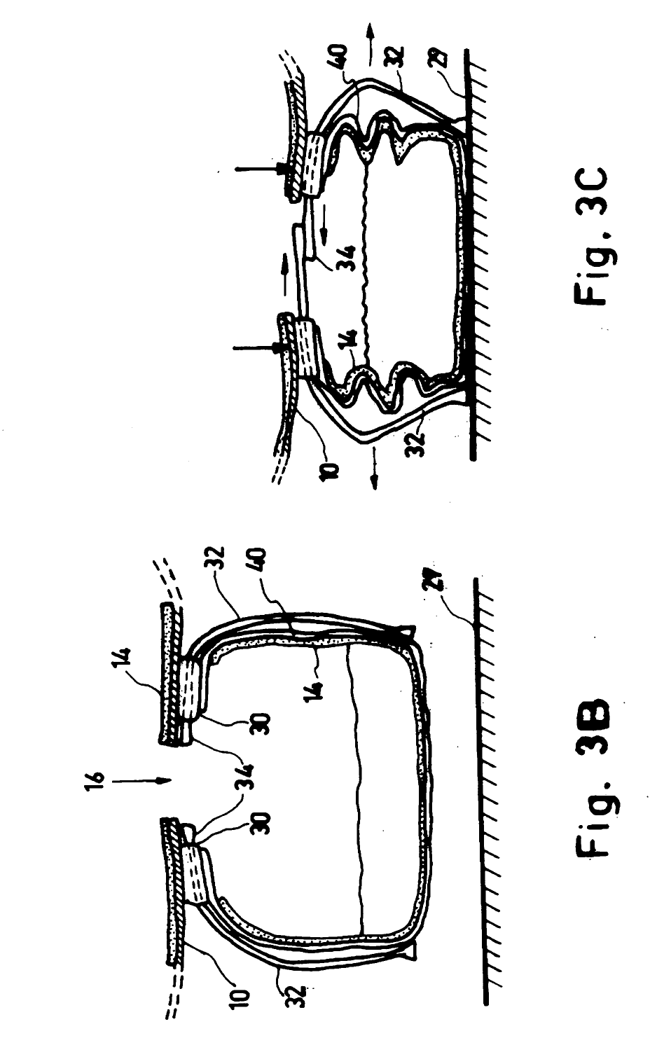

FIGS. 3A-C are various views of another embodiment of the containment means of the present invention.

In one embodiment of the invention (as in FIG. 3A), a single pair of flexible slide tracks 30 guides a movable flexible rib 32 which ends in a tongue piece 34 which slides upward along sack 40 closing opening 16 of containment means 40 whenever a wearer sits down on a rigid surface 29.

FIGS. 3B-C illustrate cross-section views of a second embodiment of the invention comprising two slide tracks 30 and two ribs 32 with tongue pieces 34 formed from an upper end thereof, oppositely disposed from one another, in relation to opening 16, so that both tongue pieces 34 meet and close opening 16 from the upward force on ribs 32 when a wearer assumes a sitting position on rigid surface 29 as shown in FIG. 3C. The closure is effected due to the vertical compression force exerted on containment means 40 between the body of the wearer and the rigid sitting surface 29 which partially collapses sack 40 which folds to retain body wastes held therein, while ribs 32 are deformed outward as shown by the horizontal arrows.

Upon resuming a standing position, the pressure on containment means 40 is relieved and due to the resiliency of the ribbing 32 containment means 40 resumes its initially opened form assisted by the expansion of semi-rigid plastic tracks 30 which produce a small, but significant counter-force to the compression, in effect, exposing the opening 16 which once again allows body wastes to collect in containment means 40 through opening 16.

The advantage of providing two tongue pieces 34 is that the length of ribs 32 can be shortened and the closing and opening action occurs over a shorter distance, thus more quickly protecting the wearer from accidental spill-out of wastes from the containment means 40 when a user of diaper 40 assumes a sitting position.

Slide tracks 30 are made of any resilient, semi-rigid material, but in a preferred embodiment of the invention, are made of plastic attached at an upper point in proximity to opening 16 to the underside of diaper 10. Slide tracks 30 restrain tongue pieces 34 which are made of the same plastic material as slide tracks 30. Tongue pieces 34 are formed as extensions of ribbing 32.

In the embodiment of the invention of FIG. 3 tongue pieces 34 extend slightly beyond the upper end of slide tracks 30 to facilitate smooth movement. Note that tongue pieces 34 do not interfere with opening 16 when in a retracted position within slide tracks 30 so as not to become soiled with body wastes passing through opening 16. When closed, tongue pieces 34, in a preferred embodiment of the invention, are protected from undesirable pollution by a thin, disposable sleeve of nylon (not shown) tightly fitted to the upper portion of tongue piece 34 making clean-up, when necessary, convenient and easy. Tongue pieces 34 alternatively may be coated with a hard plastic coating, such as polymer, which is easily washed clean and resists stain.

Optionally, a support rib can be present within the diaper for maintaining a gap between the diaper and the skin, extending the length of the diaper, as described in my previous patent U.S. Pat. No. 6,464,673. In such case, the semi-rigid ribbing upon the containment sack does not always need to be included, as long as the lowest part of the sack is fixed to the support rib present within the diaper itself. This fixing ensures the shape and placement of the sack are maintained. Optionally in such case, the closure means that ensure closure of the sack are comprised of an area of lesser rigidity upon the containment means, as compared to the rigidity found in the remainder of the containment means. Optionally the containment means may be shaped to have a narrowed neck and a containment body wider than the narrow neck.

Thus the improved anti-irritant diaper construction provides a disposable diaper which prevents waste from coming in contact with the skin of the wearer. Furthermore, because the waste is confined within a protective and separately disposable container, the diaper changing process is made easier, cleaner, and less expensive since solid wastes are hygienically isolated from the absorbent material of a diaper, making the diaper itself reusable if not wet.

Having described the invention with regard to certain specific embodiments thereof, it is to be understood that the description is not meant as a limitation, since further modifications may now suggest themselves to those skilled in the art, and it is intended to cover such modifications as fall within the scope of the appended claims.

I claim:

1. An improved, anti-irritant, disposable diaper comprised of a front portion, a crotch area, a rear portion, closing tabs for securing the diaper upon a wearer, waste containment means for collecting and isolating body wastes; attachment means for detachably attaching said containment means to the crotch area of the interior said diaper; wherein the improvement comprises:
a reinforced, semi-rigid ribbing disposed contiguous to at least one surface of said containment means, for restoring said containment means to its original shape after being compressively deformed;
closure means provided on said containment means, to ensure closure of said containment means when the wearer is seated;
and wherein an opening is provided in the crotch area of said diaper, for insertion of said containment means within said opening, and said containment means are retained within said opening by said attachment means.

2. The diaper of claim 1, wherein said closure means are regions of lesser rigidity present in said semi-rigid ribbing, which cause formation of a tight narrowing in the containment means when the wearer is seated.

3. The diaper of claim 2, wherein said regions of lesser rigidity are pre-creases along at least one crease line in said semi-rigid ribbing.

4. The diaper of claim 2, wherein said semi-rigid ribbing bends outwards during seating of a wearer, and the narrowing is formed at the neck of the containment means.

5. The diaper of claim 2, wherein said semi-rigid ribbing bends inwards during seating of a wearer, and the narrowing is formed in a position lower than the neck of the containment means.

6. The diaper of claim 1, wherein said semi-rigid ribbing is provided as a single band which transverses the height of opposing upright sides and the bottom surfaces of the containment means.

7. The diaper of claim 6, wherein the width of said semi-rigid ribbing is similar to the width of the containment means.

8. The diaper of claim 1, wherein said semi-rigid ribbing is provided as two bands each of which transverses two sides and the bottom of the containment bag, so that the bag is surrounded by a band on each upright side.

9. The diaper of claim 1, wherein said closure means are a flexible flap on an axis, extending from a portion of the crotch area of the diaper and being positioned under the wearer so as to cover the containment means when the wearer is seated and to withdraw when the wearer resumes an upright position.

10. The diaper of claim 1, wherein said closure means are at least one movable flexible rib having a tongue piece, said rib and said tongue piece being slidable within a slide track disposed upon the opening of said containment means, such that sitting of the wearer exerts pressure upon the rib and induces its movement within the slide track causing projection of the tongue piece over the opening, and whereby termination of seating results in retraction of said tongue piece from said opening.

11. The diaper of claim 10, wherein two tongue pieces are oppositely disposed from one another, so that both tongues meet to close said opening, and retract to expose said opening.

12. The diaper of claim 10, wherein said slide tracks are semi-rigid.

13. The diaper of claim 10, wherein said tongue piece is coated to allow it to be easily washed.

14. The diaper of claim 1, wherein said containment means are provided with pre-creases in the material of said containment means, for regulating folding of said containment means during seating of a wearer, into a predetermined folded shape.

15. The diaper of claim 1, additionally comprising an overflow chamber for wastes within the containment means.

16. The diaper of claim 1, additionally comprising a support rib within the diaper for maintaining a gap between the diaper and the skin.

17. An improved, anti-irritant, disposable diaper comprised of a front portion, a crotch area, a rear portion, closing tabs for securing the diaper upon a wearer, waste containment means for collecting and isolating body wastes; attachment means for detachably attaching said containment means to the crotch area of the interior said diaper; a support rib within the diaper for maintaining a gap between the diaper and the skin, wherein the improvement comprises:
closure means provided on said containment means, to ensure closure of said containment means when the wearer is seated;
and wherein an opening is provided in the crotch area of said diaper, for insertion of said containment means within said opening, and said containment means are retained within said opening by said attachment means.

18. The diaper according to claim 17, wherein said closure means are comprised of an area upon said containment means having lesser rigidity than the remainder of said containment means.

19. The diaper according to claim 17, wherein said containment means are provided with a narrowed neck and a containment body wider than said narrow neck.
